(19) United States
(12) Patent Application Publication
(10) Pub. No.: US 2011/0092535 A1
(43) Pub. Date: Apr. 21, 2011

(54) Novel Epilepsy Treatment

(76) Inventor: Nicholas Mark Barnes, Birmingham (GB)

(21) Appl. No.: 11/989,158

(22) PCT Filed: Jul. 21, 2006
(86) PCT No.: PCT/GB2006/002733

§ 371 Date: Mar. 19, 2009

(51) Int. Cl. A61K 31/439; C07D 451/14; C07D 451/06; A61K 31/46; A61P 25/08; C07D 471/18

(52) U.S. Cl. 514/294; 546/112; 546/126; 514/299; 514/304; 546/94

(57) ABSTRACT

The present invention resides in the use of a 5-HT3 receptor antagonist as medicament for the treatment of temporal lobe epilepsy associated with hippocampal sclerosis (TLE-HS) in a human patient. Tropisetron or Dolasetro are the preferred compounds.

The present invention relates to a treatment for non-drug responsive epilepsy and more particularly to temporal lobe epilepsy associated with hippocampal sclerosis (TLE-HS).

Epilepsy is a common neurological disorder where debilitating epileptic seizures arise from increased neuronal activity in the brain. Epilepsy affects approximately 40 to 50 million people worldwide. In Europe the incidence is 40 to 70 per 100,000 people and the cost to European healthcare totals [symbol] 20 billion annually. Approximately 70% of sufferers receive appropriate treatment, the remaining 30% being unresponsive or only partially responsive to current drug therapy. Current therapy for non-drug responsive epilepsy sufferers may, if appropriate, involve invasive brain surgery to remove the affected part of the brain. The cost of such surgery varies from $50,000-$200,000 per patient.

Epileptic seizures result from increased excitation of neurones in the brain. Patients with temporal lobe epilepsy associated with hippocampal sclerosis (TLE-HS) often do not respond to current drug therapy for epilepsy but require surgical resection to alleviate their symptoms. A number of neurotransmitter systems are affected in the hippocampi of patients with TLE-HS. Excitatory glutamatergic neurons within subfields of the hippocampus degenerate whereas the major inhibitory GABAergic neurons are relatively spared. However, recent evidence suggests that these normally inhibitory GABAergic neurons are excitatory in TLE (Cohen et al., Science, 298, 1418-1421, 2002). Hence, rather than reducing the neuronal excitability associated with TLE, the relative preservation of these GABAergic neurons would exacerbate the condition.

It is an object of the present invention to provide a therapy for the treatment of TLE-HS and a medicament for use in said therapy.

The present invention resides in the use of a 5-HT₃ receptor antagonist as a medicament for the treatment of temporal lobe epilepsy associated with hippocampal sclerosis (TLE-HS) in a human patient.

The present invention is based on the discovery by the inventor that in temporal lobe tissue from patients with TLE-HS, there is extensive up-regulation of 5-HT₃ receptors associated with both the glutamatergic and GABAergic neurons. This suggests the 5-HT₃ receptor as a potential therapeutic target for TLE-HS.

Balakrishnan et al. (Epilepsy & Behaviour, 1, 22-26 (2000)) suggest that a specific 5-HT₃ receptor antagonist, ondansetron, is effective in low doses against maximal electric shock-induced seizures in rats. However, the rat model is not relevant to TLE-HS in humans. For example this animal model is sensitive to phenyloin, but in human TLE, phenyloin is not adequate. In any event, there is no clear teaching as to why ondansetron achieves the observed effect. Moreover, there is a clear teaching that ondansetron is ineffective at higher doses (>2 mg/kg).

5-HT₃ receptor antagonists are widely produced as drugs for the treatment of emesis. These compounds display very few and minor side effects which would make them suitable for chronic administration.

The nature of the 5-HT₃ receptor antagonist is not particularly limited and may, for example, be selected from 5-HT₃ receptor antagonists known and commercialized for other medical conditions. Suitable 5-HT₃ receptor antagonists include Granisetron, Tropisetron and Dolasetron. Preferably, ondansetron is excluded from the scope of the invention.

Preferably the 5-HT₃ receptor antagonist is selective for the 5-HT₃ receptor.

The invention also resides in the treatment of a human patient afflicted with temporal lobe epilepsy associated with hippocampal sclerosis (TLE-HS), comprising administration of a therapeutic amount of a 5-HT₃ receptor antagonist.

The invention also resides in a pharmaceutical formulation comprising a 5-HT₃ receptor antagonist in admixture with a pharmaceutically acceptable carrier therefor.

The dosage administered to a patient will normally be determined by the prescribing physician and will generally vary according to the age, weight and response of the individual patient, as well as the severity of the patient's symptoms. However, in most instances, an effective therapeutic dosage will be in the same range as is known to be effective for the treatment of emesis, e.g. from about 0.01 mg/kg to about 0.2 mg/kg of body weight and, preferably, from 0.02 mg/kg to about 0.1 mg/kg of body weight administered in single or divided doses. In some cases, however, it may be necessary to use dosages outside these limits.

While it is possible for an active ingredient to be administered alone as the raw chemical, it is preferable to present it as a pharmaceutical formulation. The formulations of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 1 mg and 8 mg of the active ingredient. Preferably, the formulation is suitable for administration from one to six, such as two to four, times per day (twice per day being particularly convenient). Formulations suitable for nasal or buccal administration, such as the self-propelling powder-dispensing formulations described hereinafter, may comprise 0.001 to 0.2% w/w, for example about 0.01% w/w of active ingredient.

The formulations include those in a form suitable for oral, parenteral (including subcutaneous, intraperitoneal, intramuscular and intravenous), intra-articular, topical, rectal, nasal or buccal administration. Oral administration is preferred, with IV administration being preferred in severe cases.

Formulations of the present invention suitable for oral administration may be in the form of discrete units such as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution. Depending upon the level of dilution, the formulation will be either a liquid at room temperature (in the region of about 20° C.) or a low-melting solid.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient.

Formulations suitable for topical administration include transdermal devices. For example, administration can be accomplished using a patch of the reservoir and porous membrane type or of a solid matrix variety. In either case, the active agent is delivered continuously from the reservoir or microcapsules through a membrane into an active agent permeable adhesive, which is in contact with the skin of the patient. The active agent is absorbed through the skin at a controlled and predetermined rate. In the case of microcapsules, the encapsulating agent may also function as the membrane. The transdermal device will usually include the compound in a suitable solvent system with an adhesive system, such as an acrylic emulsion. The formulation may desirably include a penetrant which enhances absorption or penetration of the active ingredient through the skin. Examples of such dermal penetrants include dimethylsulfoxide and related analogues.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200 μm.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation. Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation-whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Surfactants are desirable since they prevent agglomeration of the particles of active ingredient and maintain the active ingredient in suspension. Especially valuable are liquid non-ionic surfactants and solid anionic surfactants or mixtures thereof. Suitable liquid non-ionic surfactants are those having a hydrophile-lipophile balance (HLB, see Journal of the Society of Cosmetic Chemists Vol. 1 pp. 311-326 (1949)) of below 10, in particular esters and partial esters of fatty acids with aliphatic polyhydric alcohols. The liquid non-ionic surfactant may constitute from 0.01 up to 20% w/w of the formulation, though preferably it constitutes below 1% w/w of the formulation. Suitable solid anionic surfactants include alkali metal, ammonium and amine salts of dialkyl sulphosuccinate and alkyl benzene sulphonic acid. The solid anionic surfactants may constitute from 0.01 up to 20% w/w of the formulation, though preferably below 1% w/w of the composition. Solid diluents may be advantageously incorporated in such self-propelling formulations where the density of the active ingredient differs substantially from the density of the propellant; also, they help to maintain the active ingredient in suspension. The solid diluent is in the form of a fine powder, preferably having a particle size of the same order as that of the particles of the active ingredient. Suitable solid diluents include sodium chloride, sodium sulphate and sugars.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution. Such formulations usually contain a flavouring agent such as saccharin sodium and a volatile oil. A buffering agent such as sodium metabisulphite and a surface-active agent may also be included in such a formulation which should also contain a preservative such as methylhydroxybenzoate.

Other formulations suitable for nasal administration include a powder, having a particle size of 20 to 500 microns, which is administered in the manner in which snuff is taken, i.e. by rapid inhalation through the nasal passage from a container of the powder held close up to the nose.

In addition to the aforementioned ingredients, the formulations of this invention may include one or more additional ingredients such as diluents, buffers, flavouring agents, binders, surface active agents, thickeners, lubricants, preservatives e.g. methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, e.g. Miglyol 810.

The present invention will now be exemplified with reference to the following data.

### 1. Expression of the 5-HT₃ Receptor in Human Hippocampus

### 1.1 Selection of Tissue

Resected tissue from patients with HS resulting from TLE was obtained from patients receiving therapeutic resections of temporal lobe and hippocampus. Hippocampal sclerosis was verified by neuropathological analysis of neuronal density in hippocampal sub-regions including CA 1-4 and dentate gyrus (also allowing an assessment of 5-HT₃ receptor expression relative to neuronal density). ‘Control’ tissue was obtained from patients who died without a neurological or psychiatric disorder (see Table 1 below). It is recognized that it will be difficult to ‘match’ precisely the post-mortem delay in obtaining the ‘control’ tissue to the relative speed of obtaining the resected tissue. However, in an attempt to negate differences in ‘post-mortem’ delay between the two groups, some resected brain tissue was placed in an environment to replicate as far as possible the post-mortem conditions apparent for the ‘control’ tissue.

TABLE 1
Details of patients

| | ‘Control’ patients | ‘Epileptic’ patients |
|---|---|---|
| | (n = 10) | (n = 9) |
| Age | 50 (range 19-73) yrs | 33 (range 16-55) yrs |
| Post-mortem delay^{a} | up to 21 hrs | up to 1 hr |

^{a}For epileptic tissue, post-mortem delay corresponds to the delay from surgical removal of tissue until freezing.

### 1.2 Quantification of 5-HT₃ Receptors by Autoradiography

5-HT₃ receptor binding levels were determined in the brain samples (‘control’ and TLE-HS patient) by quantitative receptor autoradiography (Barnes, J. M., Ge, J., Parker, R. M. C., Barber, P. C., Barnes, N. M. (1996) J. Neurological Sci., 144, 119-127, 1996]. Briefly, frozen hippocampal tissues were surrounded in embedding medium (OCT compound) before 20 μm sections were cut using a cryostat and thaw mounted onto gelatine-coated glass slides. Sections were stored desiccated at −80° C. until assay.

To quantify 5-HT₃ receptors, slide mounted hippocampal sections were removed from storage (−80° C.) and allowed to equilibrate to 4° C. (typically 30-60 min). To reduce endogenous levels of 5-HT in the tissue, the sections were pre-incubated for 30 min in Hepes/Krebs buffer (mM; Hepes, 50.0; NaCl. 118.0; KCl, 4.75; KH₂PO₄, 1.2; MgSO₄, 1.2; CaCl₂, 2.5; NaHCO₃, 25.0, glucose 11.0; final pH 7.4) at 4° C. The slides were then washed by briefly dipping the sections in fresh Hepes/Krebs buffer (4° C.) before being incubated in Hepes/Krebs buffer which contained 0.5 nM [³H]—(S)-zacopride (78-83 Ci/mmol) in the absence (total binding) or presence of granisetron (1 μM; non-specific binding) for 60 min at 4° C. Immediately following incubation, the tissue sections were washed in Hepes/Krebs buffer (4° C.; 2×5 min) and dipped (1 s) in distilled water (4° C.) to remove buffer salts. The sections were rapidly dried in a stream of cold dry air and exposed to Hyperfilm-[³H] (Amersham) in X-ray cassettes together with [³H]standards (Amersham) for between 6-10 weeks. Autoradiographic films were developed in Kodak LX 24 developer (5 min) and Kodak Unifix (5 min). In an attempt to improve resolution, after drying, some tissue sections were exposed to photographic emulsion (K5 or G5; Ilford), which had been dried onto cover slips. The cover slips had been permanently fixed at one end to the microscope slide supporting the tissue section such that the photographic emulsion could be developed without damaging the tissue and the position of the photographic emulsion coated coverslip during exposure could be re-established. Photographic emulsion was developed in D19 developer (Kodak; 5 min) and AL4 fixer (Kodak; 5 min). Autoradiographic films were quantified by reference to [³H]standards (fmol/mg tissue equivalent values for intact grey matter; Amersham) using image analysis (MCID, Imaging Research Inc). Only qualitative information concerning the cellular distribution of 5-HT₃ receptor binding was obtained from studies using the ‘cover slip’ technique.

### 1.3 Semi-Quantitative Expression of 5-HT₃ Receptor Subunits Assessed by Immunohistochemistry

The minimal pharmacological differences apparent between different 5-HT₃ receptor isoforms (i.e. 5-HT₃ receptors with differing subunit compositions, for instance homomeric 5-HT_{3A} and heteromeric 5-HT_{3A/3B} receptors; (Brady, C. A., Stanford, I. M., Ali, I., Lin, L., Dubin, A. E., Hope, A. G., Barnes, N. M. (2001) Neuropharmacology, 41, 282-284)) precludes the use of radioligand receptor autoradiography to determine the molecular basis for the changes in 5-HT₃ receptor expression. Hence, the additional detection of individual 5-HT₃ receptor subunit expression was assessed using immunohistochemistry techniques. A further benefit is the cellular resolution achieved with this latter technique.

The 5-HT_{3A} subunit was labelled with a selective antibody which has previously been used to label 5-HT_{3A} subunit protein in western blots and brain and gut tissue sections at the level of the light and electron microscope (Morales, M., Bloom, F. E. (1997) J. Neurosci., 17, 3157-3167; Fletcher, S., Barnes, N. M. (1997) Br. J. Pharmacol., 122, 655-662; Fletcher, S., Lindstrom J. M., McKernan, R. M., Barnes, N. M. (1998) Neuropharmacology, 37, 397-399; Michel, K., Zeller, F., Langer, R., Nekarda, H., Dover, T. J., Barnes, N. M., Schemann, M. (2005) Gastroenterology, 128, 1317-1326). The 5-HT_{3B} receptor subunit was identified using an antibody that recognises selectively the human 5-HT_{3B} receptor (see below).

Paraffin-embedded human temporal lobe sections (5 μm) incorporating the hippocampus were cut using a microtome and mounted on glass microscope slides. The tissue sections were rehydrated from xylene, through ethanol, to water and then incubated in 0.3% hydrogen peroxide to quench endogenous peroxidase activity before incubation in PBS (1 hr, room temperature; buffer changed every 10 min) followed by incubation (1 hr, room temperature) in PBS plus Triton-X-100 (TX100; 0.3%) and normal goat serum (3%). For the immunohistochemical detection of 5-HT₃ receptor subunits, antibodies against the 5-HT_{3A} subunit or the 5-HT_{3B} subunit were used. As both antibodies were generated in rabbits, it was not possible to perform double labelling experiments. The tissue was incubated in PBS/TX100 and normal goat serum (3%) containing the primary antibodies. After washing in PBS/TX100, the tissue was incubated with biotinylated goat anti-rabbit IgG (1:200; Vector) for 2 hrs prior to washing and incubation with ABC reagent (Vector) for 2 hrs. After washing, immunoreactivity was visualised with the peroxidase substrate diaminobenzidine (0.025%), which generated a brown reaction product. Tissue was then dehydrated through rising ethanol concentrations and then xylene and mounted to allow visualisation of immunoreactive cells (the brain sections were also stained lightly with haematoxylin to aid identification of structures).

1.3a Generation of the Selective Polyclonal Antibody that Recognises Selectively the Human 5-HT_{3B} Subunit

A peptide sequence (18 amino acids) was selected from the cloned amino acid sequence of the human 5-HT_{3B} receptor subunit sequence (amino acids 341-358 of the h5-HT_{3B} protein sequence). This peptide sequence showed no significant homology to other known cloned receptors or mammalian proteins when screened using the GenEMBL gene database and SwissProt database, and was identified as having good immunogenicity.

A polyclonal antibody was raised in three New Zealand White SPF rabbits to this hapten following conjugation through a terminal cysteine to keyhole limpet haemocyanin (KLH) using the cross-linking agent m-maleimomidobenzoic acid N-hydroxysuccimide ester. Following removal of pre-immune serum, the animals were immunised with the peptide in Freund's complete adjuvant for the first two injections (14 days apart), followed by a further five fortnightly booster injections of peptide in Freund's incomplete adjuvant in order to maximise the titre of the anti-sera. Test bleeds were carried out regularly and screened using enzyme-linked immunosorbent assay (ELISA) to monitor antibody titre. All animals responded to immunisation producing peptide specific antibodies. One week after the final injection, animals were anaesthetized and the final bleed performed via cardiac puncture. After each bleed, blood was allowed to clot and clots to retract at 4° C. overnight. Serum was aspirated and any suspended cells allowed to settle prior to final separation. Sodium azide was added to all sera to a final concentration of 0.1% w/v. All pre and post-immune sera were stored at −80° C. until required.

Initial experiments demonstrated that sera displayed high titres to recognise the antigenic peptide from the human 5-HT_{3B} subunit (assessed by ELISA) and more importantly, displayed desirable characteristics when assessed for the ability to recognise, selectively, the h5-HT_{3B} subunit by SDS-PAGE/Western blotting and immunocytochemistry.

### 1.4 Phenotyping of 5-HT₃ Receptor Subunit Expressing Neurones

Cells which express 5-HT₃ subunit-like immunoreactivity can be phenotyped using standard immunohistochemical techniques. Initially, selective markers for the principal neurotransmitter associated with the neurone should be investigated (i.e. glutamate and GABA). Further phenotyping can then be undertaken to define expression by sub-populations of neurons (e.g. GABA neurones based on their differential expression of neuropeptides and Ca²⁺ binding proteins e.g. see Freund, T. F., Buzsaki, G. (1996) Hippocampus, 6, 347-470).

1.5 Expression of 5-HT₃ Receptor Subunit mRNA Assessed by In Situ Hybridization and RT-PCR

5-HT₃ receptor subunit expression was confirmed using in situ hybridization and RT-PCR (to detect mRNA transcripts; for detailed methodology see Parker, R. M. C., Barnes, N. M. (1998) mRNA: detection by in situ and northern hybridisation. In: Receptor binding techniques (Keen, K., Ed), Humana Press Inc., pp 239-273). With appropriate controls, these techniques offer higher degrees of selectivity compared to immunohistochemistry and hence confirmation of 5-HT₃ subunit expression at both the cellular (in situ hybridization) and regional (RT-PCR) level adding confidence to the results.

In the absence of an antibody that recognizes, selectively, the 5-HT_{3C} receptor subunit, the in situ hybridization and RT-PCR techniques are the only approach allowing assessment of 5-HT_{3C} subunit expression. Likewise for the 5-HT_{3D} and 5-HT_{3E} receptor subunits, although at least in ‘normal’ human brain tissue, these subunits have yet to be detected.

1.6 Pharmacology of the 5-HT₃ Receptor Expressed in Hippocampus from ‘Control’ and TLE-HS Patients.

Human hippocampus from patients that had died within 48 hrs from a non-neurological and non-psychiatric disorder (‘control’ patients) or resected from patients with TLE-HS were snap frozen at −80° C. To form the radioligand binding homogenate, tissue was thawed in buffer (50 mM Tris, pH7.4) before homogenisation at 4° C. using a Polytron blender. The homogenate was then centrifuged (48,000×g, 10 min, 4° C.) and the resultant pellet resuspended in buffer and re-centrifuged. The radioligand binding homogenate was prepared by resuspension of the pellet in buffer at a concentration of 100 mg original wet weight of tissue per 1 ml. Radioligand binding assays were performed similar to our methodology described previously (Bufton et al., Neuropharmacology, 32, 1325-1331, 1993). Briefly, tubes in triplicate contained 150 μl of competing drug or vehicle (50 mM Tris, pH7.4; total binding) and 100 μl of [³H]-granisetron (≈1-2 nM; NEN, 82 Ci mmol⁻¹). An aliquot (250 μl) of the appropriate tissue homogenate was added to initiate binding, which was allowed to proceed at room temperature for 90 min before termination by rapid filtration and washing under vacuum through Whatman GF/B filters, followed by assay of the radioactivity remaining on the filters.

### 1.7 Semi-Quantitative Expression of SERT Assessed by Immunohistochemistry

Paraffin-embedded human hippocampal sections (5 μm) from either ‘control’ patients or resected tissue from patients with TLE-HS were cut using a microtome and mounted on glass microscope slides. The tissue sections were rehydrated from xylene, through ethanol, to water and then incubated in 0.3% hydrogen peroxide to quench endogenous peroxidase activity before incubation in PBS (1 hr, room temperature; buffer changed every 10 min) followed by incubation (1 hr, room temperature) in PBS plus Triton-X-100 (TX100; 0.3%) and normal bovine serum (10%). For the immunohistochemical detection of the 5-HT transporter, SERT, the tissue was incubated in PBS/TX100 and normal bovine serum (10%) containing the primary monoclonal antibody ST-51 (dilution of the supplied antibody 1:5000; obtained from the commercial antibody supplier MAb Technologies [www.mabtechnologies.com]) overnight at 4° C. In some experiments, the monoclonal antibody was pre-incubated with the immunizing peptide to prevent interaction of the antibody with SERT (‘peptide block’)). After washing in PBS/TX100, the tissue was incubated with biotinylated horse anti-mouse IgG (1:300; Vector) for 2 hrs prior to washing and incubation with ABC reagent (Vector) for 2 hrs. After washing, immunoreactivity was visualised with the peroxidase substrate diaminobenzidine (0.025%), which generated a brown reaction product. Tissue was then dehydrated through rising ethanol concentrations and then xylene and mounted to allow visualisation of immunoreactive cells (the brain sections were also stained lightly with haematoxylin to aid identification of structures).

### 2. Experimental Results

The experimental results will now be described with reference to the accompanying drawings in which:—

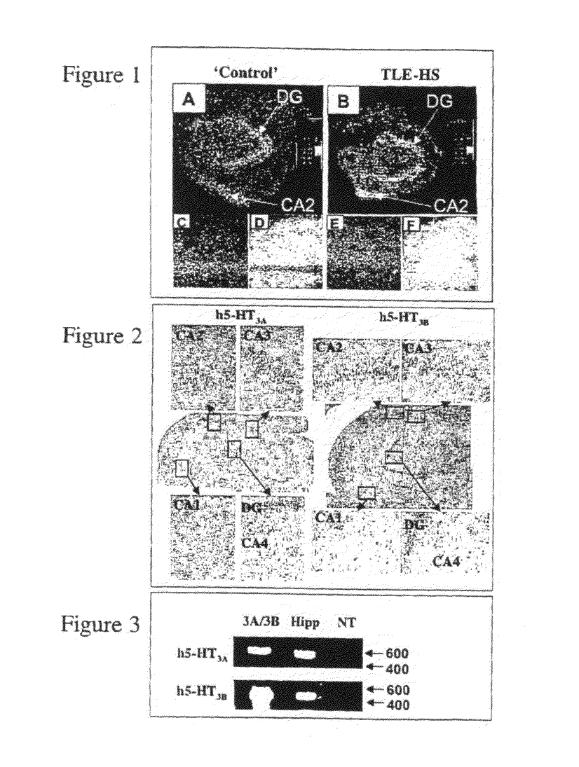

FIG. 1 shows expression of the 5-HT₃ receptor in human hippocampus from ‘control’ patients and patients with TLE-HS assessed by receptor autoradiography;

Panels A and B; Pseudo-colour autoradiograms of [³H]—(S)-zacopride binding to hippocampus from ‘control’ and epileptic (TLE-HS) patients respectively;
Panels C and E; ‘Cover slip’ dark-field autoradiograms of the hippocampal dentate gyrus (DG) from ‘control’ and epileptic (TLE-HS) patients respectively;
Panels D and F; Histological staining of control and TLE-HS tissue corresponding to Panels C and E respectively,

FIG. 2. shows expression of the 5-HT₃ receptor in human hippocampus from ‘control’ patients using immunohistochemistry to locate the h5-HT_{3A} and h5-HT_{3B} receptor subunits, and

FIG. 3. shows expression of the 5-HT₃ receptor in human hippocampus from ‘control’ patients using RT-PCR to detect the h5-HT_{3A} and h5-HT_{3B} subunit mRNA.

### 2.1 5-HT₃ Receptor Distribution and Expression Levels in ‘Control’ Hippocampus Quantified by Receptor Autoradiography

Within ‘control’ human hippocampal sections, the levels of specific [³H]—(S)-zacopride binding sites displayed a marked regional heterogeneity (Table 2; FIG. 2). Highest densities of specific binding (defined by the inclusion of granisetron, 1 μM) were detected in the dentate gyrus (DG) and the CA2 field (Table 2; FIG. 1, Panel A), with lower levels of specific binding in other hippocampal fields and adjacent brain regions. Throughout the hippocampal formation, levels of non-specific [³H]—(S)-zacopride binding (defined by the presence of granisetron, 1.0 μM) were barely observable above background.

TABLE 2
Specific [³H]-(S)-zacopride (0.5 nM) binding levels in
various regions of the human hippocampus and adjacent brain
regions from patients with temporal lobe epilepsy with hippocampal
sclerosis (TLE-HS) and ‘control’ patients (non-specific
binding defined by the inclusion of granisetron; 1.0 μM).

| | Specific [³H]-(S)- | |
|---|---|---|
| | zacopride binding (fmol/mg) | |
| | ‘Control’ | ‘TLE-HS’ |
| Brain region | patients* | patients* |
| Dentate gyrus (granule cell layer) | 7.34 ± 1.20 | 7.28 ± 0.90 |
| Dentate gyrus (molecular layer) | 2.60 ± 0.48 | 5.57 ± 0.70* |
| Hippocampal CA1 field | 1.38 ± 0.33 | 2.35 ± 0.62 |
| Hippocampal CA2 field | 4.72 ± 0.49 | 10.32 ± 1.11 |
| Hippocampal CA3 field | 3.19 ± 0.46 | 3.16 ± 0.76 |
| CA4 (dentate gyrus hilar region) | 2.83 ± 0.41 | 2.08 ± 0.47 |

*Data represents mean ± S.E.M., n = 9-10.

Use of the ‘cover slip’ technique allowed the distribution of [³H]—(S)-zacopride binding to be viewed in direct comparison with individual cells within the hippocampal formation. Within the dentate gyrus, [³H]—(S)-zacopride binding displayed a distinct pattern with highest levels overlying the cell bodies of the granule cells (FIG. 1, Panel C). Within the CA2 field of the hippocampus, [³H]—(S)-zacopride binding was associated with the pyramidal cell layer.

### 2.2 5-HT₃ Receptor Distribution and Expression Levels in ‘Epileptic’ Hippocampus Quantified by Receptor Autoradiography

Hippocampal tissues from patients with epilepsy displayed a different pattern of specific [³H]—(S)-zacopride distribution compared to ‘control’ patients. Within the dentate gyrus, the levels of specific [³H]—(S)-zacopride displayed little change (Table 2), however, the width of the band had markedly increased (FIG. 1, Panel B) and use of the ‘cover slip’ technique indicated that the binding had spread to additionally encompass the full width of the molecular cell layer (FIG. 1, Panel E). A similar observation can be made by comparison of Panels D and F of FIG. 1 (note the distinct granule cell layer, (denoted by a bracket in panel D, is only evident in the tissue from the ‘control’ patient). Within the CA2 field of the hippocampus, the specific [³H]—(S)-zacopride remained in association with the pyramidal cells (FIG. 1, Panel B), however, the levels of binding were increased approximately two fold (Table 2).

The relative distribution of specific [³H]—(S)-zacopride binding within ‘epileptic’ tissue that had been subjected to conditions intended to mimic the post-mortem conditions encountered by the ‘control’ tissue was comparable to the ‘epileptic’ tissue that had been processed immediately following resection.

### 2.3 5-HT₃ Receptor Subunit Expression in ‘Control’ Hippocampus Assessed by Immunohistochemistry

Immunohistochemistry was performed with paraffin-embedded hippocampal sections from human brain using a range of primary antibody concentrations (1:50-1:500). Under optimum conditions, specific h5-HT_{3A} and h5-HT_{3B} subunit-immunoreactivity were clearly evident in pyramidal neurones in all hippocampal CA fields, although for both antibodies, immunoreactivity was qualitatively lower within the pyramidal neurones of the CA1 region (FIG. 2). For both antibodies, immunoreactivity was also associated with the large neurons (displaying the morphology of GABAergic interneurones) within the CA4 (hilus field; FIG. 2). Faint immunoreactivity for both antibodies was associated with the granule cells of the dentate gyrus (FIG. 2).

When the primary antibody was replaced with non-immune rabbit serum at an identical dilution to the immunoreactive sera, and tissues subsequently processed in an identical manner, no immunoreactivity was apparent in adjacent sections of human hippocampus (data not shown).

### 2.4 5-HT₃ Receptor Subunit Expression in ‘Epileptic’ Hippocampus Assessed by Immunohistochemistry

Relatively high levels of h5-HT_{3A} and h5-HT_{3B}-immunoreactivity were associated with the surviving pyramidal neurones associated with the CA2 field of the hippocampus. This data shows that the surviving neurones in the epileptic tissue express the 5-HT3A and 5-HT3B subunits—the expression also appears relatively high compared to the control tissue.

### 2.5 5-HT₃ Receptor Subunit Expression in ‘Control’ Hippocampus Assessed by RT-PCR

In addition to using antibodies to demonstrate expression of the 5-HT_{3A} and 5-HT_{3B} subunits within the human hippocampal tissue, RT-PCR using specific primers for either the h5-HT_{3A} or h5-HT_{3B} subunit transcripts generated specific products of the appropriate size when using templates derived from the human hippocampal samples (Hipp) or the HEK 293 cell line heterologously expressing the subunits (3A/3B). No product were generated in the control which was absent of template (NT) (FIG. 3).

2.6 Pharmacology of the 5-HT₃ Receptor Expressed in Hippocampus from ‘Control’ and “Epileptic” Patients

Drugs competed for the [³H]-granisetron binding site in both ‘control’ and TLE-HS hippocampal homogenates at similar concentrations giving rise to similar pKi values (Table 3), that were also consistent with the effective concentrations of the drugs to interact with the 5-HT₃ receptor in human putaman determined previously (Table 3). Values in the table represent the mean pKi value from three independent experiments (the percentage standard error of the mean pKi value was less than 2%). This data demonstrates that the 5-HT3 receptor expressed by the epileptic tissue has a near identical pharmacology to the receptor expressed in control tissue. This is important as it strongly indicates that drugs that have been developed to work on ‘normal’ 5-HT3 receptors will work in the present invention

TABLE 3
Pharmacology of the [³H]-granisetron binding site in ‘control’
hippocampus and that from patients with TLE-HS.

| | pKi | | |
|---|---|---|---|
| | ‘Control’ | TLE-HS | |
| Competing drug | hippocampus | hippocampus | Putamen¹ |
| (S)-Zacopride | 8.93 | 8.97 | 8.96 |
| Granisetron | 8.27 | 8.20 | 8.03 |
| Tropisetron | 8.13 | 8.07 | 7.89 |
| Ondansetron | 7.93 | 7.90 | 7.76 |
| 5-Hydroxytryptamine | 6.80 | 6.73 | 6.80 |

¹The pharmacology of the [³H]-granisetron binding site described previously in human putamen (Bufton et al., Neuropharmacology, 32, 1325-1331, 1993).

### 2.7 Semi-Quantitative Expression of SERT Assessed by Immunohistochemistry

Specific SERT-immunoreactivity was clearly evident throughout all hippocampal CA fields as fine fibers (characteristic of 5-HT neurones) in both the ‘control’ and TLE-HS tissue. Although this procedure is only semi-quantitative, it was consistently observed that the density of SERT-immunopositive fibres was higher in the hippocampal tissue arising from patients with TLE-HS in comparison to the ‘control’ hippocampal tissue. This provides evidence that the 5-HT neuronal innervation of the hippocampus of patients with TLE-HS is at least as rich as that in ‘control’ hippocampus and may be higher, indicating that the concentration of 5-HT activating the 5-HT₃ receptor in patients with TLE-HS is at least comparable, and probably higher, than occurs in ‘control’ hippocampus. If the 5-HT levels had been reduced, it might have been possible that the observed 5-HT3 receptor density had increased in an attempt to compensate for the reduction in 5-HT levels (the 5-HT transporter (SERT) is a phenotypic marker for 5-HT neurones). For the purposes of the present invention, the 5-HT3 receptors need to be activated in the epileptic tissue since if they are not activated by 5-HT, then an antagonist (e.g. Granisetron) would be ineffective.

When the primary antibody was pre-incubated with the immunizing peptide (‘peptide block’) and then used at an identical dilution to the monoclonal antibody that had not been pre-incubated with immunizing peptide, and tissues subsequently processed in an identical manner, no immunoreactivity was apparent in adjacent sections of human hippocampus (data not shown).

### 3. Phase II Clinical Trial

Approximately ten adult patients accessing the Queen Elizabeth Hospital (University Hospital Birmingham NHS Foundation Trust) with drug resistant temporal lobe epilepsy who are scheduled for resection of the epileptic tissue will be admitted for two days for clinical observation and EEG telemetry. Once baseline condition of the patient has been evaluated, a 5-HT₃ receptor antagonist (granisetron 1 mg b.d.) will be added to any ongoing medication for a period of three months. The patients will keep seizure diaries and will be followed up as outpatients (weekly for the first month, and then monthly for further two months). In the follow up, patients will be reviewed clinically, their seizure diaries checked along with blood tests for liver function at 0, 1, 4, 8 and 12 weeks.

1. The use of a 5-HT₃ receptor antagonist as a medicament for the treatment of temporal lobe epilepsy associated with hippocampal sclerosis (TLE-HS) in a human patient.

2. The use of claim 1, wherein the 5-HT₃ receptor antagonist is Granisetron, Tropisetron or Dolasetron.

3. The use of claim 1, wherein the 5-HT₃ receptor antagonist is selective for the 5-HT₃ receptor.

4. A method of treating a human patient afflicted with temporal lobe epilepsy associated with hippocampal sclerosis (TLE-HS), comprising administration of a therapeutic amount of a 5-HT₃ receptor antagonist.

5. The method of claim 4, wherein the effective therapeutic amount is from 0.01 mg/kg to about 0.2 mg/kg of body weight.

6. (canceled)

7. The method of claim 4, wherein said treatment is oral or parenteral.

8. The method of claim 5, wherein said treatment is oral or parenteral.
